(19) United States
(12) Patent Application Publication
(10) Pub. No.: US 2024/0390244 A1
(43) Pub. Date: Nov. 28, 2024

(54) COMPOSITION COMPRISING AN OXIDATIVE DYE SALT

(71) Applicant: KAO CORPORATION, Tokyo (JP)

(72) Inventors: Hartmut MOEHRING, Darmstadt (DE); Takahito NAKAMURA, Tokyo (JP)

(73) Assignee: KAO CORPORATION, Tokyo (JP)

(21) Appl. No.: 18/696,633

(22) PCT Filed: Sep. 29, 2022
(86) PCT No.: PCT/EP2022/077126

§ 371 Date: Mar. 28, 2024

(30) Foreign Application Priority Data
Sep. 30, 2021 (EP) 21200129.1

(51) Int. Cl. A61K 8/41; A45D 19/00; A61K 8/24; A61K 8/25; A61Q 5/10

(52) U.S. Cl. CPC A61K 8/411; A45D 19/0066; A61K 8/24; A61K 8/25; A61K 8/415; A61Q 5/10; A61K 2800/4324

(57) ABSTRACT

An oxidative dyeing composition A for keratin fibers, including a) a salt of 1,4-diamino-2-methoxymethyl-benzene, and c) an alkalizing agent which is at least one selected from inorganic alkalizing agents, organic alkalizing agents, salts thereof, or mixtures thereof. The salt of 1,4-diamino-2-methoxymethyl-benzene is at least one selected from a sulfate salt, a hydrogen sulfate salt, a hydrochloride salt, a hydrobromide salt, a nitrate salt, a phosphate salt, a hydrogen phosphate salt, a dihydrogen phosphate salt, a methosulfate salt, a citrate salt, a succinate salt, a tartrate salt, a lactate salt, a tosylate salt, a benzenesulfonate salt, an acetate salt, or mixtures thereof.

### FIELD OF THE INVENTION

The present invention relates to a composition for dyeing of keratin fibers with an oxidative dye salt. Furthermore, a two-part composition, kit-of-parts and dyeing method is disclosed.

### BACKGROUND OF THE INVENTION

Oxidative dyes possess superior dyeing ability and grey coverage, but their packaging and formulation need careful consideration. Once exposed to oxygen and humidity, they may react and form colored intermediates. In such a case, similar to the process on head, the dyeing composition becomes dark.

Cosmetic industry has tackled these problems by excluding air oxygen from the compositions. For example, many dyeing compositions are individually packaged or, if offered as multi-use device, supplied with a protective gas such as nitrogen or carbon dioxide.

A particular challenge is the stable formulation of dyeing products with low amount of water.

As the prior art has not satisfactorily solved the above challenges, there is a real need to develop dyeing compositions, which are stable over time.

### SUMMARY OF THE INVENTION

The first object of the present invention is an oxidative dyeing composition A for keratin fibers, preferably human keratin fibers, more preferably human hair, comprising
a) one or more salt(s) of 1,4-diamino-2-methoxymethyl-benzene,
c) one or more alkalizing agent(s) selected from inorganic or organic alkalizing agent(s), and/or their salt(s), and/or their mixtures,
wherein the one or more salt(s) of 1,4-diamino-2-methoxymethyl-benzene is/are a sulfate salt, a hydrogen sulfate salt, a hydrochloride salt, a hydrobromide salt, a nitrate salt, a phosphate salt, a hydrogen phosphate salt, a dihydrogen phosphate salt, a methosulfate salt, a citrate salt, a succinate salt, a tartrate salt, a lactate salt, a tosylate salt, a benzenesulfonate salt, an acetate salt, and/or their mixtures,
wherein the one or more inorganic alkalizing agent(s) as compound(s) according to group c) is one or more metasilicate(s), disilicate(s), carbonate(s), bicarbonate(s), and/or phosphate(s), and/or their metal salts such as alkali or earth alkaline salts, and/or their mixtures,
wherein the one or more organic alkalizing agent(s) and/or its/their salt(s) as compound(s) according to group c) is one or more ammonium salt(s), one or more organic alkyl and/or alkanol amines and/or their salt(s) according to the following general structure:

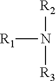

wherein R₁, R₂, and R₃ are independently selected from H, linear C₁-C₆ alkyl which may be substituted with one hydroxyl group, or branched C₃-C₁₂ alkyl or alkanol, wherein at least one of R₁, R₂, or R₃ is different from H, and/or their salts, and/or their mixtures, and
wherein the total concentration of water is 10% by weight or less, calculated to the total weight of composition A.

The second object of the present invention is a two-part hair dyeing composition or three-part hair dyeing and bleaching composition comprising composition A as defined above and an aqueous composition B, optionally having a pH in the range of 1 to 6 and optionally comprising one or more oxidizing agent(s), preferably hydrogen peroxide, and optionally a bleaching composition, wherein compositions A and/or B comprise(s) one or more oxidizing agent(s).

The third object of the present invention is a method for dyeing keratin fibers, preferably human keratin fibers, more preferably human hair, comprising the steps of:
i) providing composition A as defined above and mixing it with an aqueous composition B as defined above to yield a ready-to-use composition having a pH in the range of 7 to 12,
ii) applying the ready-to-use composition onto keratin fibers and leaving it for a time period in the range of 1 min to 60 min,
iii) rinsing-off the keratin fibers and optionally drying the keratin fibers.

The fourth object of the present invention is a use of one or more salt(s) of 1,4-diamino-2-methoxymethyl-benzene for stabilizing solid dyeing compositions comprising 4-chlororesorcinol, 2-methylresorcinol, 1,3-bis-(2,4-diamino-phenoxy)-propane, 2,4-diaminophenoxyethanol, 2-methyl-5-amino-6-chlorphenol, 2-amino-4-hydroxyethylaminoanisole, 5-amino-6-chloro-o-cresol, 2-amino-3-hydroxypyridine, 3-amino-2,4-dichlorophenol, 2,4,5,6-tetraaminopyrimidine, 2,5,6-triamino-5-pyrimidinol, and/or their salt(s), and/or their mixtures, wherein the one or more salt(s) of 1,4-diamino-2-methoxymethyl-benzene is/are a sulfate salt, a hydrogen sulfate salt, a hydrochloride salt, a hydrobromide salt, a nitrate salt, a phosphate salt, a hydrogen phosphate salt, a dihydrogen phosphate salt, a methosulfate salt, a citrate salt, a succinate salt, a tartrate salt, a lactate salt, a tosylate salt, a benzenesulfonate salt, an acetate salt, and/or their mixtures.

A fifth object of the present invention is a use of composition A as defined above for dyeing of keratin fibers, preferably human keratin fibers, more preferably human hair.

### DETAILED DESCRIPTION OF THE INVENTION

Inventors of the present invention have unexpectedly found out that composition A according to the independent claims has superior dyeing intensity independently of its storage conditions. In addition, it delivers a superior dyeing stability upon preparation of the ready-to-use mixture.

### Composition for Dyeing

The present invention is directed to an oxidative dyeing composition A for keratin fibers, preferably human keratin fibers, more preferably human hair, comprising
a) one or more salt(s) of 1,4-diamino-2-methoxymethyl-benzene,
c) one or more alkalizing agent(s) selected from inorganic or organic alkalizing agent(s), and/or their salt(s), and/or their mixtures,
wherein the one or more salt(s) of 1,4-diamino-2-methoxymethyl-benzene is/are a sulfate salt, a hydrogen sulfate salt, a hydrochloride salt, a hydrobromide salt, a nitrate salt, a phosphate salt, a hydrogen phosphate salt, a dihydrogen phosphate salt, a methosulfate salt, a citrate salt, a succinate salt, a tartrate salt, a lactate salt, a tosylate salt, a benzenesulfonate salt, an acetate salt, and/or their mixtures,
wherein the one or more inorganic alkalizing agent(s) as compound(s) according to group c) is one or more metasilicate(s), disilicate(s), carbonate(s), bicarbonate(s), and/or phosphate(s), and/or their metal salts such as alkali or earth alkaline salts, and/or their mixtures,
wherein the one or more organic alkalizing agent(s) and/or its/their salt(s) as compound(s) according to group c) is one or more ammonium salt(s), one or more organic alkyl and/or alkanol amines and/or their salt(s) according to the following general structure:

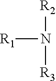

wherein R₁, R₂, and R₃ are independently selected from H, linear C₁-C₆ alkyl which may be substituted with one hydroxyl group, or branched C₃-C₁₂ alkyl or alkanol, wherein at least one of R₁, R₂, or R₃ is different from H, and/or their salts, and/or their mixtures, and
wherein the total concentration of water is 10% by weight or less, calculated to the total weight of composition A.

It is preferred from the viewpoint of stability that the salts of compound(s) according to group a) is/are a sulfate salt and/or a hydrochloride salt, and/or their mixtures, preferably it is a sulfate salt.

It is preferred from the viewpoint of dyeing intensity that the total concentration of compound(s) according to group a) is 0.01% by weight or more, preferably in the 0.05% by weight or more, further more preferably 0.1% by weight or more, still more preferably 0.5% by weight or more, still further more preferably 1% by weight or more, calculated to the total weight of composition A.

It is preferred from the viewpoint of cosmetic safety that the total concentration of compound(s) according to group a) is 65% by weight or less, preferably 50% by weight or less, further more preferably 35% by weight or less, still further more preferably 30% by weight or less, still further more preferably 20% by weight or less, calculated to the total weight of composition A.

For attaining the above-mentioned effects, it is preferred that the total concentration of compound(s) according to group a) is in the range of 0.01% to 65% by weight, preferably in the range of 0.05% to 50% by weight, more preferably in the range of 0.1% to 35% by weight, still more preferably in the range of 0.5% to 30% by weight, still further more preferably in the range of 1% to 20% by weight, calculated to the total weight of composition A.

It is further preferred from the viewpoint of stability that the total concentration of water in composition A is 5% by weight or less, preferably 1% by weight or less, more preferably 0.1% by weight or less, calculated to the total weight of composition A, still more preferably composition A is anhydrous.

The term ‘anhydrous’ denotes a composition that is free of added water. However, this does not exclude bound water such as residual moisture or crystal water.

It is preferred from the viewpoint of color shading that composition A comprises one or more oxidative dye precursor or oxidative dye coupler different from group a) as compound(s) according to group b), preferably 5-amino-2-methylphenol, 2-methyl-5-hydroxyethylaminophenol, 2,4-diaminophenoxyethanol, 2-amino-4-hydroxyethylaminoanisol, 2-methyl-5-amino-6-chlorphenol, 1,3-bis-(2,4-diaminophenoxy)-propane, 2-bis(2-hydroxyethyl)-aminotoluene, 2-amino-5-methylphenol, resorcinol, 2-methylresorcinol, 4-chlororesorcinol, 2-amino-4-chlorophenol, 5-amino-4-methoxy-2-methylphenol, 2-aminophenol, 3-amino-phenol, 1-methyl-2-hydroxy-4-aminobenzene, 3-N,N-dimethyl aminophenol, 2,6-dihydroxy-3,5-dimethoxypyridine, 5-amino-3-methylphenol, 6-amino-3-methylphenol, 2,4-diaminophenoxyethanol, 1,3-diamino-benzene, 1-amino-3-(2′-hydroxyethylamino)benzene, 1-amino-3-[bis(2′-hydroxy-ethyl) amino]benzene, α-naphthol, 4,6-dichlororesorcinol, 1,3-diamino-toluene, 4-hydroxy-1,2-methylenedioxy benzene, 1,5-dihydroxy naphthalene, 1,6-dihydroxy naphthalene, 1,7-dihydroxy naphthalene, 2,7-dihydroxy naphthalene, 1-hydroxy-2-methyl naphthalene, 4-hydroxy-1,2-methyldioxy benzene, 2,4-diamino-3-chlorophenol, 5-amino-2-methoxyphenol and/or 1-methoxy-2-amino-4-(2′-hydroxyethyl amino)-benzene, and/or their salt(s), and/or their mixtures, preferably one or more compound(s) according to group b) is/are selected from 4-chlororesorcinol, 2-methylresorcinol, 1,3-bis-(2,4-diaminophenoxy)-propane, 2-methyl-5-amino-6-chlorphenol, 2-amino-3-hydroxypyridine, 5-amino-6-chloro-o-cresol, 3-amino-2,4-dichlorophenol, and/or their salt(s), and/or their mixtures, more preferably 1,3-bis-(2,4-diamino-phenoxy)-propane, 2,4-diaminophenoxyethanol, 2-amino-4-hydroxyethylaminoanisole, 5-amino-6-chloro-o-cresol, 3-amino-2,4-dichlorophenol, and/or their salt(s), and/or their mixtures.

It is preferred from the viewpoint of dyeing intensity that the total concentration of compound(s) according to group b) is 0.01% by weight or more, preferably in the 0.05% by weight or more, further more preferably 0.08% by weight or more, calculated to the total weight of composition A.

It is preferred from the viewpoint of cosmetic safety that the total concentration of compound(s) according to group b) is 20% by weight or less, preferably 15% by weight or less, further more preferably 10% by weight or less, still further more preferably 5% by weight or less, still further more preferably 3.5% by weight or less, calculated to the total weight of composition A.

For attaining the above-mentioned effects, it is preferred that the total concentration of compound(s) according to group b) is in the range of 0.01% to 20% by weight, preferably in the range of 0.05% to 15% by weight, more preferably in the range of 0.08% to 10% by weight, still more preferably in the range of 0.08% to 5% by weight, still further more preferably in the range of 0.08% to 3.5% by weight, calculated to the total weight of composition A.

It is further preferred from the viewpoint of dyeing intensity that the total concentration of compound(s) according to groups a) and b) is 0.1% by weight or more, preferably 0.5% by weight or more, more preferably 1% by weight or more, calculated to the total weight of composition A.

It is further preferred from the viewpoint of cosmetic safety that the total concentration of compound(s) according to groups a) and b) is 60% by weight or less, preferably 50% by weight or less, more preferably 35% by weight or less, still more preferably 20% by weight or less, calculated to the total weight of composition A.

For attaining the above-mentioned effects, it is preferred that the total concentration of compound(s) according to groups a) and b) is in the range of 0.1% to 60% by weight, preferably in the range of 0.5% to 50% by weight, more preferably in the range of 1% to 35% by weight, still more preferably in the range of 1% to 20% by weight, calculated to the total weight of composition A.

It is preferred from the viewpoint of color shades that the weight ratio of compound(s) according to group a) to compound(s) according to group b) is 0.2 or more, more preferably 0.3 or more, still more preferably 0.5 or more.

It is preferred from the viewpoint of color shades that the weight ratio of compound(s) according to group a) to compound(s) according to group b) is 5 or less, more preferably 3 or less, still more preferably 2 or less.

For attaining the above-mentioned effects, it is preferred that the weight ratio of compound(s) according to group a) to compound(s) according to group b) is in the range of 0.2 to 5, preferably in the range of 0.3 to 3, more preferably in the range of 0.5 to 2.

Composition A comprises one or more inorganic or organic alkalizing agent(s), and/or their salt(s), and/or their mixtures as compound(s) according to group c).

One or more inorganic alkalizing agent(s) of composition A as compound(s) according to group c) is/are one or more metasilicate(s), disilicate(s), carbonate(s), bicarbonate(s), and/or phosphate(s), and/or their metal salts such as alkali or earth alkaline salts, and/or their mixtures. The preferred inorganic alkalizing agents is/are sodium metasilicate, trisodium phosphate and/or tripotassium phosphate, and/or their mixtures, more preferably it/they is/are trisodium phosphate and/or tripotassium phosphate.

In addition, or alternatively, composition A comprises one or more organic alkalizing agent(s) and/or its/their salt(s) as compound(s) according to group c). The organic alkalizing agent(s) is/are one or more ammonium salt(s), one or more organic alkyl and/or alkanol amines and/or their salt(s) according to the following general structure:

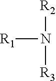

wherein R₁, R₂, and R₃ are independently selected from H, linear C₁-C₆ alkyl which may be substituted with one hydroxyl group, or branched C₃-C₁₂ alkyl or alkanol, wherein at least one of R₁, R₂, or R₃ is different from H, and/or their salts, and/or their mixtures.

It is preferred from the viewpoint of dyeing intensity that one or more organic alkalizing agent(s) as compound(s) according to group c) is/are selected from alkyl and/or alkanolamine(s) and/or its/their salt(s), more preferably they/it is selected from monoethanolamine, diethanolamine, monoethanol methylamine, monoethanol dimethylamine, diethanolmethylamine, monoethanolethylamine, monoethanoldiethylamine, diethanolethylamine, monoethanolpropylamine, monoethanoldipropylamine, diethanolpropylamine, monoethanolbutylamine, diethanolbutylamine, trimethylamine, triethylamine, 2-amino-2-methylpropanol, tris-(hydroxymethyl)-aminomethane and/or its/their salt(s), and/or their mixtures, from the viewpoint of providing alkalinity and cosmetic safety as well as their low odor.

Preferably, one or more ammonium salt is/are ammonium sulfate, ammonium phosphate, ammonium chloride.

The most preferred inorganic or organic alkalizing agents as compound(s) according to group c) is/are selected from sodium metasilicate, trisodium phosphate, tripotassium phosphate, monoethanolamine, 2-amino-2-methylpropanol, tris-(hydroxymethyl)-aminomethane, and/or its/their salt(s), ammonia and or its salt(s), and/or their mixtures, from the viewpoint of providing alkalinity and cosmetic safety.

It is preferred from the viewpoint of dyeing intensity that the total concentration of compound(s) according to group c), preferably of inorganic or organic alkalizing agents, is 1% by weight or more, more preferably 2% by weight or more, further more preferably 5% by weight or more, calculated to the total weight of composition A.

It is preferred from the viewpoint of cosmetic safety that the total concentration of compound(s) according to group c), preferably of inorganic or organic alkalizing agents, is 80% by weight or less, more preferably 70% by weight or less, further more preferably 60% by weight or less, calculated to the total weight of composition A.

For attaining the above-mentioned effects, it is preferred that the total concentration compound(s) according to group c), preferably of inorganic or organic alkalizing agents, is in the range 1% to 80% by weight, preferably in the range of 2% to 70% by weight, more preferably in the range of 5% to 60% by weight, calculated to the total weight of composition A.

### Direct Dyes

It is further preferred from the viewpoint of dyeing intensity the composition A comprises one or more direct dye(s).

Suitably, one or more direct dye(s) may be HC Blue 18, HC Red 18, HC Yellow 16, Disperse Black 9, Acid Yellow 1, 2-amino-6-chloro-4-nitrophenol, and/or their salt(s), and/or their mixtures.

Suitably, the total concentration of direct dye(s) is 0.01% by weight or more, preferably 0.05% by weight or more, more preferably 0.1% by weight or more, calculated to the total weight of composition A.

Suitably, the total concentration of direct dyes is 20% by weight or less, preferably 15% by weight or less, more preferably 10% by weight or less, calculated to the total weight of composition A.

Suitably, the total concentration of direct dyes is in the range of 0.01% to 20% by weight is in the range of 0.05% to 15% by weight, more preferably in the range of 0.1% to 10% by weight, calculated to the total weight of composition A.

### Compound(s) According to Group d)

It is preferred from the viewpoint of composition stability and convenience of use that composition A comprises one or more pulverulent excipient as compound(s) according to group d).

The term ‘excipient’ denotes a compound, which may act as filling material and dispersant for the other compounds of composition A and does not react with the dyes or the alkalizing agent, and, thus, confer the powder a high degree of storage stability over an extended period of time.

Composition A of the present invention may comprise an organic and/or an inorganic pulverulent excipient in which dyes are dispersed.

Suitable one or more pulverulent excipient is/are selected from diatomaceous earth, kaolin, bentonite, silicates, starches such as corn starch, tapioca starch, rice starch, wheat starch and potato starch, nylon powder, montmorillonit, gypsum, sawdust and perlite, and/or their mixtures, more preferably it is corn starch, from the viewpoint of stability.

Preferably, the total concentration of compound(s) according group d), more preferably the total concentration of organic and/or an inorganic pulverulent excipients, is 50% by weight or more, more preferably 55% by weight or more, further more preferably 60% by weight or more, still further more preferably 65% by weight or more, even further more preferably 70% by weight or more, even more preferably 75% by weight or more, calculated to the total weight of composition A, from the viewpoint of achieving good dispersability of the direct dyes in the powder and quick dissolution of the powder.

Preferably, the total concentration of compound(s) according group d), more preferably the total concentration of organic and/or an inorganic pulverulent excipients, is 98% by weight or less, more preferably 95% by weight or less, further more preferably 90% by weight or less, calculated to the total weight of composition A, from the viewpoint of achieving good dispersability of the dyes in the powder and formulation freedom.

For attaining the above mentioned effects, it is preferred that the total concentration of compound(s) according group d), more preferably the total concentration of organic and/or an inorganic pulverulent excipients, is in the range of 50% to 98% by weight, more preferably in the range of 55% to 95% by weight, further more preferably in the range of 60% to 90% by weight, still further more preferably in the range of 65% to 90% by weight, still further more preferably in the range of 70% to 90% by weight, even more preferably in the range of 75% to 90% by weight, calculated to the total weight of composition A.

### Powder Composition

In one aspect of the present invention, composition A may be a solid composition, preferably a powder composition.

The term ‘powder’ denotes a solid composition at 25° C. and atmospheric pressure. The term relates to freely flowing powders as well as compressed powders such as tablets. The powder composition may also comprise water as long as its nature of the solid state at 25° C. is unchanged. Depending on the type of powder, a water content of 10% by weight or less, calculated to the total weight of composition A, may be acceptable. This does not exclude the presence of residual moisture from air or crystal water bound to ingredients. Preferably, composition A is an anhydrous powder composition, from the viewpoint of stability.

### Lipophilic Compounds as Compounds According to Group e)

Compositions A and/or B may comprise one or more lipophilic compound(s) as compound(s) according to group e).

Preferably, compounds according to group e) are selected from C₁₂ to C₂₂ fatty alcohols, esters of C₃ to C₂₂ alcohols with C₁₂ to C₂₂ fatty acids, C₈ to C₂₂ fatty acids, vegetable oils, and/or silicones, and/or hydrocarbon-based products, and/or their mixtures, from the viewpoint of cosmetic compatibility.

Suitable C₁₂ to C₂₂ fatty alcohols are are myristyl alcohol, cetyl alcohol, stearyl alcohol, behenyl alcohol, and cetearyl alcohol.

Suitable esters of C₃ to C₂₂ alcohols with C₁₂ to C₂₂ fatty acids are isopropyl myristate, isopropyl palmitate, and myristyl myristate.

Suitable C₈ to C₂₂ fatty acids are oleic acid, linoleic acid, and palmitic acid.

Suitable vegetable oils are olive oil, almond oil, sunflower oil, and argan oil.

Suitable silicones are non-aminated and/or aminated silicones. The latter are commonly known as amodimethicones.

It is preferred from the viewpoint of forming a stable composition and user friendliness that the total concentration of compounds according to group e) is 1% by weight or more, more preferably 2% by weight or more, further more preferably 3% by weight or more, calculated to the total weight of each of the compositions A and/or B.

It is preferred from the viewpoint of forming a stable composition that the total concentration of compounds according to group e) is 20% by weight or less, more preferably 15% by weight or less, further more preferably 12% by weight or less, calculated to the total weight of each of the compositions A and/or B.

For attaining the above-mentioned effects, the total concentration of compounds according to group e) is in the range of 1% to 20% by weight, preferably in the range of 2% to 15% by weight, more preferably in the range of 3% to 12% by weight, calculated to the total weight of each of the compositions A and/or B.

### Liquid Composition

In another aspect of the present invention, composition A of the present invention is a liquid composition at 25° C. and atmospheric pressure comprising one or more organic solvent(s) as compound(s) according to group f). Preferably, composition A is anhydrous, from the viewpoint of dye stability.

The term ‘liquid’ denotes a physical state at 25° C. and atmospheric pressure, i.e., composition A is liquid at room temperature.

The term ‘anhydrous’ denotes a composition A, which is free of added water. This does not exclude the presence of residual moisture from air or crystal water bound to ingredients.

For this aspect of the present invention, composition A may comprise one or more organic solvent(s).

The organic solvent(s) may be selected to dissolve the dyes. Preferred solvents are mono-, di-, and trivalent alcohols and/or their mixtures.

Preferred mono-, di-, and trivalent alcohols from the viewpoint of cosmetic safety and dissolution capacity are ethanol, n-propanol, isopropanol, propylene glycol, ethylene glycol, benzyl alcohol, phenoxyethanol, and glycerol, and/or their mixtures.

It is further preferred from the viewpoint of solution stability that the total concentration of organic solvents is 75% by weight or more, more preferably 80% by weight or more, further more preferably 85% by weight or more, calculated to the total weight of composition A.

It is further preferred from the viewpoint of dyeing intensity that the total concentration of organic solvents is 98% by weight or less, more preferably 95% by weight or less, further more preferably 92% by weight or less, calculated to the total weight of composition A.

For attaining the above-mentioned effects, it is preferred that the total concentration of organic solvents is in the range of 75% to 98% by weight, more preferably 80% to 95% by weight, further more preferably in the range of 85% to 92% by weight, calculated to the total weight of composition A.

### Hair Dyeing Oil

In one aspect of the present invention, composition A is a hair dyeing oil.

Preferably, composition A has a paste-like viscosity, more preferably having a viscosity in the range of 10,000 mPas to 500,000 mPas, more preferably in the range of 20,000 mPas to 400,000 mPas, still more preferably in the range of 30,000 mPas to 250,000 mPas, measured by cone-plate viscometry at 25° C. and atmospheric pressure, preferably with a Brookfield viscometer and spindle #4 at 10 rpm for 1 min at 25° C. and atmospheric pressure.

For this purpose, that the total concentration of one or more compound(s) according to group e) is 90% by weight or less, preferably in the range of 50% to 90% by weight, calculated to the total weight of composition A.

Preferably, the hair dyeing oil composition A is anhydrous, from the viewpoint of dye stability.

### Surfactants as Compounds According to g)

Compositions A and/or B may further comprise one or more surfactant(s) as compound according to group g), preferably selected from non-ionic surfactants, anionic surfactants, cationic surfactants, and/or amphoteric/zwitterionic surfactants, and/or their salt(s), and/or their mixtures, more preferably selected from anionic surfactants and/or their salt(s), from the viewpoint of stabilizing the composition and improving wettability and mixability.

Preferably, the anionic surfactants may be selected from ethoxylated or non-ethoxylated alkyl ether sulfate surfactants, alkyl sulfates, ethoxylated and/or non-ethoxylated alkyl carboxylates, ethoxylated or non-ethoxylated amino acid surfactants, and/or their mixtures, and/or their salts.

Suitable examples are alkyl sulfate or preferably ethoxylated alkyl ether sulfate surfactants or mixtures thereof, and/or salts thereof, having an alkyl chain length of C₁₀ to C₂₂ and an ethoxylation degree from 1 to 50.

Suitable non-ionic surfactants may be selected from alkyl polyglycosides, ethoxylated triglycerides, ethoxylated fatty alcohols, ethoxylated fatty acid esters, and/or their mixtures.

Suitable cationic surfactants are quaternary ammonium surfactants having a carbon chain length in the range of C₁₂ to C₂₂ or surfactants having a tertiary amine group and at least one alkyl chain having a carbon chain length in the range of C₁₂ to C₂₂ such as alkylamidoalkylamine surfactants, and/or their salts. Suitable examples are cetrimonium chloride and behentrimonium chloride.

Suitable amphoteric/zwitterionic surfactants are of betaine type. Suitable compounds may be selected from alkyl betaines and/or alkylamido betaines. A preferred compound selected from alkyl betaines is lauryl betaine. A preferred compound selected from alkylamido betaines is cocamidopropyl betaine. The disclosure also relates to the salts of the compounds.

Suitable concentration ranges for surfactants are in the range of 0.1% to 10% by weight, calculated to the total weight of each of the compositions A and/or B, from the viewpoint of enhancing wettability of keratin fibers, physical stability, and mixability with other compositions.

### Thickening Polymers

From the viewpoint of cosmetic safety, it is further preferred that the compositions A and/or B comprise one or more thickening polymer.

Composition A and/or B may comprise one or more thickening polymer(s) selected from non-ionic thickening polymers and/or anionic thickening polymers, and/or their mixtures.

Preferably, the thickening polymers are selected from polymers resulting in an aqueous solution and/or aqueous dispersion at pH ranges between 7 and 12 having a viscosity of at least 1,000 mPa·s measured at a polymer concentration of 1% by weight in water at 25° C. determined by cone-plate viscometry at 25° C. under atmospheric conditions, calculated to the total weight of the composition, for examples measured with a Brookfield viscometer at 10 rpm for 1 min, with spindle #4 at 25° C. and atmospheric pressure.

Suitable non-ionic thickening polymers are cellulose-based polymers. Suitable examples of cellulose-based polymers are methylcellulose, hydroxymethylcellulose, hydroxyethylcellulose, hydroxypropylcellulose, hydroxyethyl-methylcellulose, and alkylated hydroxyl celluloses such as (C₂-C₅)-alkylcelluloses or cetyl hydroxyethylcellulose.

Suitable anionic thickening polymers are selected from naturally-based anionic polymers and/or synthetic anionic polymers.

Suitably, the natural anionic polymer(s) may be selected from xanthan gum, dehydroxanthan gum, hydroxypropylxanthan gum, carboxymethyl cellulose and starch based polymers such as vegetable starch and/or their synthetically modified derivatives such as hydroxypropyl starch phosphate. Equally suitable are alginic acids, sodium alginates, ammonium alginates, calcium alginates, gum arabic, and guar gum.

Suitable synthetic anionic polymers are associative thickening polymers, such as acrylates/steareth-30 methacrylate copolymer.

The preferred thickening polymer for the composition of the present invention are natural anionic polymers, more preferably xanthan gum and/or dehydroxanthan gum, from the viewpoint of their biodegradability and low environmental impact.

Preferably, the total concentration of thickening polymers of the present invention is 0.1% by weight or more, more preferably 0.25% by weight or more, more preferably 0.5% by weight or more, calculated to the total weight of each of the compositions A and/or B, from the viewpoint of providing sufficient viscosity to the composition.

Preferably, the total concentration of thickening polymers of the present invention is 15% by weight or less, more preferably 12% by weight or more, further more preferably 10% by weight or less, calculated to the total weight of each of the compositions A and/or B, from the viewpoint of providing sufficient viscosity to the composition and cost of goods.

For attaining the above-mentioned effects, it is preferred that the total concentration of thickening polymers in the composition of the present invention is in the range of 0.1% to 15% by weight, preferably in the range of 0.25% to 12% by weight, more preferably in the range of 0.5% to 10% by weight, calculated to the total weight of each of the compositions A and/or B.

It is preferred from the viewpoint of cosmetic safety that the composition of the present invention, in case that it is aqueous, has a viscosity in the range of 1,000 mPas to 25,000 mPas, preferably 2,000 mPas to 20,000 mPas, more preferably in the range of 2,500 mPas to 17,500 mPas, determined by cone plate viscometry at 25° C. under atmospheric conditions. A suitable viscometer is a Brookfield viscometer with spindle #4 measured at 10 rpm for 1 min at 25° C. and atmospheric pressure.

### Oxidizing Agents

Compositions A and/or B may comprise one or more oxidizing agent(s), preferably hydrogen peroxide.

Preferably, composition A comprises one or more oxidizing agent being solid at 25° C. and atmospheric pressure, more preferably one or more peroxide being solid at 25° C. and atmospheric pressure, still more preferably it comprises a metal peroxide such as sodium peroxide, potassium peroxide, magnesium peroxide, melamine peroxide, urea peroxide, and/or their mixtures.

It is further preferred from the viewpoint of dyeing performance that the total concentration of one or more oxidizing agent being solid at 25° C. and atmospheric pressure is 0.1% by weight or more, more preferably 0.25% by weight or more, further more preferably 1% by weight or more, calculated to the total weight of compositions A.

It is further preferred from the viewpoint of product performance and user safety that the total concentration of one or more oxidizing agent being solid at 25° C. and atmospheric pressure is 50% by weight or less, more preferably 40% by weight or less, further more preferably 30% by weight or less, calculated to the total weight of composition A.

For attaining the above-mentioned effects, it is preferred that the total concentration of one or more oxidizing agent being solid at 25° C. and atmospheric pressure is in the range of 0.1% to 50% by weight, more preferably in the range of 0.25% to 40% by weight, further more preferably in the range of 1% to 30% by weight, calculated to the total weight compositions A.

The preferred oxidizing agent for composition B is hydrogen peroxide, from the viewpoint of dyeing performance.

It is further preferred from the viewpoint of dyeing performance that the concentration of oxidizing agents in compositions B is 0.1% by weight or more, more preferably 0.25% by weight or more, further more preferably 1% by weight or more, calculated to the total weight of compositions B.

It is further preferred from the viewpoint of product performance and user safety that the concentration oxidizing agents in composition B is 20% by weight or less, more preferably 15% by weight or less, further more preferably 12% by weight or less, calculated to the total weight of the composition B.

For attaining the above-mentioned effects, it is preferred that the concentration of oxidizing agents in composition B is in the range of 0.1% to 20% by weight, more preferably in the range of 0.25% to 15% by weight, further more preferably in the range of 1% to 12% by weight, calculated to the total weight of composition B.

### Two-Part or Three-Part Composition

The present invention is also directed to a two-part hair dyeing composition or three-part hair dyeing and bleaching composition comprising composition A as defined above and an aqueous composition B, optionally having a pH in the range of 1 to 6 and optionally comprising one or more oxidizing agent(s), preferably hydrogen peroxide, and optionally a bleaching composition, wherein compositions A and/or B comprise(s) one or more oxidizing agent(s).

Preferably, the pH of the composition is measured with a glass electrode at 25° C. and atmospheric pressure.

In case composition A of the present invention as defined above already comprises one or more oxidizing agents, the composition B may be free of oxidizing agents.

In case composition A does not comprise oxidizing agents, it is preferred from the viewpoint of dyeing intensity that the composition B has a pH in the range of 1 to 6 and comprises one or more oxidizing agent(s), preferably hydrogen peroxide.

Preferably, from the viewpoint of stability, the composition B has a pH in the range of 1.5 to 5, further more preferably in the range of 2 to 4.5 Composition B preferably is an emulsion, thickened gel, or a combination thereof, from the viewpoint of cosmetic safety as well as user friendliness. It may comprise lipophilic compound(s) according to group f) and/or surfactant(s) according to group g) and/or one or more thickening polymer(s) as defined above.

Composition B may further comprise one or more oxidizing agent(s). The preferred oxidizing agent is hydrogen peroxide.

Suitable concentration ranges for hydrogen peroxide in composition B is/are in the range of 0.1% to 20% by weight, more preferably in the range of 0.25% to 15% by weight, further more preferably in the range of 1% to 12% by weight, calculated to the total weight of the composition B.

Composition B may comprise one or more lipophilic compound(s) as compound(s) according to group f), as defined for the composition of the present invention above.

It is preferred from the viewpoint of forming a stable composition and user friendliness that the total concentration of compounds according to group f) is 1% by weight or more, more preferably 2% by weight or more, further more preferably 3% by weight or more, calculated to the total weight of composition B.

It is preferred from the viewpoint of forming a stable composition that the total concentration of compounds according to group f) is 20% by weight or less, more preferably 15% by weight or less, further more preferably 12% by weight or less, calculated to the total weight of composition B.

For attaining the above-mentioned effects, the total concentration of compounds according to group f) is in the range of 1% to 20% by weight, preferably in the range of 2% to 15% by weight, more preferably in the range of 3% to 12% by weight, calculated to the total weight of composition B.

Composition B may further comprise one or more surfactant(s) as compound according to group g), as defined above for the composition A of the present invention above.

Suitable concentration ranges for surfactants are in the range of 0.1% to 10% by weight, calculated to the total weight of composition B, from the viewpoint of enhancing wettability of keratin fibers, physical stability, and mixability with other compositions.

From the viewpoint of cosmetic safety, it is further preferred that composition B comprises one or more thickening polymer, as defined for the composition of the present invention above.

Preferably, the total concentration of thickening polymers in composition B is 0.1% by weight or more, more preferably 0.25% by weight or more, more preferably 0.5% by weight or more, calculated to the total weight of composition B, from the viewpoint of providing sufficient viscosity to composition B.

Preferably, the total concentration of thickening polymers in composition B is 15% by weight or less, more preferably 12% by weight or more, further more preferably 10% by weight or less, calculated to the total weight of composition B, from the viewpoint of providing sufficient viscosity to composition B and cost of goods.

For attaining the above-mentioned effects, it is preferred that the total concentration of thickening polymers in composition B is in the range of 0.1% to 15% by weight, preferably in the range of 0.25% to 12% by weight, more preferably in the range of 0.5% to 10% by weight, calculated to the total weight of composition B.

It is preferred from the viewpoint of cosmetic safety that composition B has a viscosity in the range of 1,000 mPas to 25,000 mPas, preferably 2,000 mPas to 20,000 mPas, more preferably in the range of 2,500 mPas to 17,500 mPas, determined by cone plate viscometry at 25° C. under atmospheric conditions. A suitable viscometer is a Brookfield viscometer with spindle #4 measured at 10 rpm for 1 min at 25° C. and atmospheric pressure.

The bleaching composition comprises one or more bleaching compound(s), preferably one or more persalt(s) and/or one or more peroxysalt(s), more preferably it comprises them at a total concentration in the range of 1% to 80% by weight, calculated to the total weight of the bleaching composition.

### Method for Dyeing

The present invention is also directed to a method for dyeing keratin fibers, preferably human keratin fibers, more preferably human hair, comprising the steps of:
i) providing composition A as defined above and optionally mixing it with the composition B as defined above to yield a ready-to-use composition having a pH in the range of 7 to 12,
ii) applying the composition A or the ready-to-use composition onto keratin fibers and leaving it for a time period in the range of 1 min to 60 min,
iii) rinsing-off the keratin fibers and optionally drying the keratin fibers.

If composition A comprises oxidizing agents, then composition B may be free of oxidizing agents.

The ready-to-use composition is applied to keratin fibers and preferably left for a time period of 1 min to 60 min as defined in step ii). Further preferred time ranges for step ii) are 5 min to 45 min, more preferred ranges are 10 min to 35 min, from the viewpoint of sufficiently dyeing.

Optionally, heat may be applied while leaving the composition A or the ready-to-use composition onto keratin fibers. Suitable temperature ranges are 30° C. to 50° C.

### Use

The present invention is also directed to a use of one or more salt(s) of 1,4-diamino-2-methoxymethyl-benzene for stabilizing solid dyeing compositions comprising 4-chlororesorcinol, 2-methylresorcinol, 1,3-bis-(2,4-diamino-phenoxy)-propane, 2,4-diaminophenoxyethanol, 2-methyl-5-amino-6-chlorphenol, 2-amino-4-hydroxyethylaminoanisole, 5-amino-6-chloro-o-cresol, 2-amino-3-hydroxypyridine, 3-amino-2,4-dichlorophenol, 2,4,5,6-tetraaminopyrimidine, 2,5,6-triamino-5-pyrimidinol, and/or their salt(s), and/or their mixtures, wherein the one or more salt(s) of 1,4-diamino-2-methoxymethyl-benzene is/are a sulfate salt, a hydrogen sulfate salt, a hydrochloride salt, a hydrobromide salt, a nitrate salt, a phosphate salt, a hydrogen phosphate salt, a dihydrogen phosphate salt, a methosulfate salt, a citrate salt, a succinate salt, a tartrate salt, a lactate salt, a tosylate salt, a benzenesulfonate salt, an acetate salt, and/or their mixtures.

The present invention is also directed to a use of composition A as defined above for dyeing of keratin fibers, preferably human keratin fibers, more preferably human hair.

The present disclosure is also directed to <1> an oxidative dyeing composition A for keratin fibers, preferably human keratin fibers, more preferably human hair, comprising
a) one or more salt(s) of 1,4-diamino-2-methoxymethyl-benzene,
c) one or more alkalizing agent(s) selected from inorganic or organic alkalizing agent(s), and/or their salt(s), and/or their mixtures,
wherein the one or more salt(s) of 1,4-diamino-2-methoxymethyl-benzene is/are a sulfate salt, a hydrogen sulfate salt, a hydrochloride salt, a hydrobromide salt, a nitrate salt, a phosphate salt, a hydrogen phosphate salt, a dihydrogen phosphate salt, a methosulfate salt, a citrate salt, a succinate salt, a tartrate salt, a lactate salt, a tosylate salt, a benzenesulfonate salt, an acetate salt, and/or their mixtures,
wherein the one or more inorganic alkalizing agent(s) as compound(s) according to group c) is one or more metasilicate(s), disilicate(s), carbonate(s), bicarbonate(s), and/or phosphate(s), and/or their metal salts such as alkali or earth alkaline salts, and/or their mixtures,
wherein the one or more organic alkalizing agent(s) and/or its/their salt(s) as compound(s) according to group c) is one or more ammonium salt(s), one or more organic alkyl and/or alkanol amines and/or their salt(s) according to the following general structure:

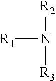

wherein R₁, R₂, and R₃ are independently selected from H, linear C₁-C₆ alkyl which may be substituted with one hydroxyl group, or branched C₃-C₁₂ alkyl or alkanol,
wherein at least one of R₁, R₂, or R₃ is different from H, and/or their salts, and/or their mixtures, and
wherein the total concentration of water is 10% by weight or less, calculated to the total weight of composition A.

<2> The composition according to clause <1> characterized in that one or more compound(s) according to group a) is/are a sulfate salt and/or a hydrochloride salt, and/or their mixtures, more preferably it is a sulfate salt.

<3> The composition according to any of the clauses <1> to <2> characterized in that the total concentration of compound(s) according to group a) is in the range of 0.01% to 65% by weight, preferably in the range of 0.05% to 50% by weight, more preferably in the range of 0.1% to 35% by weight, still more preferably in the range of 0.5% to 30% by weight, still further more preferably in the range of 1% to 20% by weight, calculated to the total weight of composition A.

<4> The composition according to any of the clauses <1> to <3> characterized in that the total concentration of water in composition A is 5% by weight or less, preferably 1% by weight or less, more preferably 0.1% by weight or less, calculated to the total weight of composition A, still more preferably composition A is anhydrous.

<5> The composition according to any of the clauses <1> to <4> characterized in that composition A comprises one or more oxidative dye precursor or oxidative dye coupler different from the ones according to group a) as compound(s) according to group b), preferably 5-amino-2-methylphenol, 2-methyl-5-hydroxyethylaminophenol, 2,4-diaminophenoxyethanol, 2-amino-4-hydroxyethylaminoanisol, 2-methyl-5-amino-6-chlorphenol, 1,3-bis-(2,4-diaminophenoxy)-propane, 2-bis(2-hydroxyethyl)-aminotoluene, 2-amino-5-methylphenol, resorcinol, 2-methylresorcinol, 4-chlororesorcinol, 2-amino-4-chlorophenol, 5-amino-4-methoxy-2-methylphenol, 2-aminophenol, 3-amino-phenol, 1-methyl-2-hydroxy-4-aminobenzene, 3-N,N-dimethyl aminophenol, 2,6-dihydroxy-3,5-dimethoxypyridine, 5-amino-3-methylphenol, 6-amino-3-methylphenol, 2,4-diaminophenoxyethanol, 1,3-diamino-benzene, 1-amino-3-(2′-hy-droxyethylamino)benzene, 1-amino-3-[bis(2′-hydroxy-ethyl) amino]benzene, α-naphthol, 4,6-dichlororesorcinol, 1,3-diamino-toluene, 4-hydroxy-1,2-methylenedioxy benzene, 1,5-dihydroxy naphthalene, 1,6-dihydroxy naphthalene, 1,7-dihydroxy naphthalene, 2,7-dihydroxy naphthalene, 1-hydroxy-2-methyl naphthalene, 4-hydroxy-1,2-methyldioxy benzene, 2,4-diamino-3-chlorophenol, 5-amino-2-methoxyphenol and/or 1-methoxy-2-amino-4-(2′-hydroxyethyl amino)-benzene, and/or their salt(s), and/or their mixtures, preferably one or more compound(s) according to group b) is/are selected from 4-chlororesorcinol, 2-methylresorcinol, 1,3-bis-(2,4-diaminophenoxy)-propane, 2-methyl-5-amino-6-chlorphenol, 2-amino-3-hydroxypyridine, 5-amino-6-chloro-o-cresol, 3-amino-2,4-dichlorophenol, and/or their salt(s), and/or their mixtures, more preferably 1,3-bis-(2,4-diamino-phenoxy)-propane, 2,4-diaminophenoxyethanol, 2-amino-4-hydroxyethylaminoanisole, 5-amino-6-chloro-o-cresol, 3-amino-2,4-dichlorophenol, and/or their salt(s), and/or their mixtures.

<6> The composition according to any of the clauses <1> to <5> characterized in that the total concentration of compound(s) according to group b) is in the range of 0.01% to 20% by weight, preferably in the range of 0.05% to 15% by weight, more preferably in the range of 0.08% to 10% by weight, still more preferably in the range of 0.08% to 5% by weight, still further more preferably in the range of 0.08% to 3.5% by weight, calculated to the total weight of composition A.

<7> The composition according to any of the clauses <1> to <6> characterized in that the total concentration of compound(s) according to groups a) and b) is in the range of 0.1% to 60% by weight, preferably in the range of 0.5% to 50% by weight, more preferably in the range of 1% to 35% by weight, still more preferably in the range of 1% to 20% by weight, calculated to the total weight of composition A.

<8> The composition according to any of the clauses <1> to <7> characterized in that the weight ratio of compound(s) according to group a) to compound(s) according to group b) is in the range of 0.2 to 5, preferably in the range of 0.3 to 3, more preferably in the range of 0.5 to 2.

<9> The composition according to any of the clauses <1> to <8> characterized in that one or more inorganic alkalizing agent(s) as compound(s) according to group c), is/are sodium metasilicate, trisodium phosphate and/or tripotassium phosphate, and/or their mixtures, more preferably it/they is/are trisodium phosphate and/or tripotassium phosphate.

<10> The composition according to any of the clauses <1> to <9> characterized in that one or more organic alkalizing agent(s) and/or its/their salt(s) as compound(s) according to group c) is/are is selected from monoethanolamine, diethanolamine, monoethanol methylamine, monoethanol dimethylamine, diethanolmethylamine, monoethanolethylamine, monoethanoldiethylamine, diethanolethylamine, monoethanolpropylamine, monoethanoldipropylamine, diethanolpropylamine, monoethanolbutylamine, diethanolbutylamine, trimethylamine, triethylamine, 2-amino-2-methylpropanol, tris-(hydroxymethyl)-aminomethane and/or its/their salt(s), and/or their mixtures, preferably they are selected from monoethanolamine, 2-amino-2-methylpropanol, tris-(hydroxymethyl)-aminomethane, and/or its/their salt(s), ammonia and or its salt(s), and/or their mixtures.

<11> The composition according to any of the clauses <1> to <8> characterized in that one or more inorganic or organic alkalizing agent(s) as compound(s) according to group c) is selected from sodium metasilicate, trisodium phosphate, tripotassium phosphate, monoethanolamine, 2-amino-2-methylpropanol, tris-(hydroxymethyl)-aminomethane, and/or its/their salt(s), and/or ammonia and or its salt(s), and/or their mixtures.

<12> The composition according to any of the clauses <1> to <11> characterized in that the total concentration of compound(s) according to group c), preferably of inorganic or organic alkalizing agents, is in the range 1% to 80% by weight, preferably in the range of 2% to 70% by weight, more preferably in the range of 5% to 60% by weight, calculated to the total weight of composition A.

<13> The composition according to any of the clauses <1> to <12> characterized in that it comprises one or more direct dye(s).

<14> The composition according to clause <13> characterized in that one or more direct dye(s) is/are HC Blue 18, HC Red 18, HC Yellow 16, Disperse Black 9, Acid Yellow 1, 2-amino-6-chloro-4-nitrophenol, and/or their salt(s), and/or their mixtures.

<15> The composition according to any of the clauses <13> to <14> characterized in that the total concentration of direct dye(s) is in the range of 0.01% to 20% by weight is in the range of 0.05% to 15% by weight, more preferably in the range of 0.1% to 10% by weight, calculated to the total weight of composition A.

<16> The composition according to any of the clauses <1> to <15> characterized in that composition A comprises one or more pulverulent excipient as compound(s) according to group d).

<17> The composition according to clause <16> characterized in that one or more compound(s) according to group d) is/are one or more organic and/or one or more inorganic pulverulent excipient(s).

<18> The composition according to any of the clauses <16> to <17> characterized in that one or more compound(s) according to group d) is/are selected from diatomaceous earth, kaolin, bentonite, silicates, starches such as corn starch, tapioca starch, rice starch, wheat starch and potato starch, nylon powder, montmorillonit, gypsum, sawdust and perlite, and/or their mixtures, more preferably it is corn starch.

<19> The composition according to any of the clauses <16> to <18> characterized in that the total concentration of compound(s) according group d), more preferably the total concentration of organic and/or an inorganic pulverulent excipients, is in the range of 50% to 98% by weight, more preferably in the range of 55% to 95% by weight, further more preferably in the range of 60% to 90% by weight, still further more preferably in the range of 65% to 90% by weight, still further more preferably in the range of 70% to 90% by weight, even more preferably in the range of 75% to 90% by weight, calculated to the total weight of composition A.

<20> The composition according to any of the clauses <1> to <19> characterized in that it is a solid composition at 25° C. and atmospheric pressure, preferably a powder composition or a pellet composition or a tablet composition, more preferably a hair dyeing powder composition or a hair dyeing pellet composition or a hair dyeing tablet composition, still more preferably it is a hair dyeing powder composition.

<21> The composition according to any of the clauses <1> to <20> characterized in that it comprises one or more lipophilic compound(s) as compound(s) according to group e), preferably selected from C₁₂ to C₂₂ fatty alcohols, esters of C₃ to C₂₂ alcohols with C₁₂ to C₂₂ fatty acids, C₈ to C₂₂ fatty acids, vegetable oils, and/or silicones, and/or hydrocarbon-based products, and/or their mixtures, from the viewpoint of cosmetic compatibility.

<22> The composition according to clause <21> characterized in that one or more C₁₂ to C₂₂ fatty alcohol(s) as compound(s) according to e) is/are myristyl alcohol, cetyl alcohol, stearyl alcohol, behenyl alcohol, and cetearyl alcohol, one or more esters of C₃ to C₂₂ alcohols with C₁₂ to C₂₂ fatty acids as compound(s) according to e) is/are isopropyl myristate, isopropyl palmitate, and myristyl myristate, one or more C₈ to C₂₂ fatty acids as compound(s) according to e) is/are oleic acid, linoleic acid, and palmitic acid, one or more vegetable oil(s) as compound(s) according to e) is/are olive oil, almond oil, sunflower oil, and argan oil, one or more silicone(s) as compound(s) according to e) is/are non-aminated and/or aminated silicones, and/or their mixtures.

<23> The composition according to any of the clauses <21> to <22> characterized in that the total concentration of compounds according to group e) is in the range of 1% to 20% by weight, preferably in the range of 2% to 15% by weight, more preferably in the range of 3% to 12% by weight, calculated to the total weight of composition A.

<24> The composition according to any of the clauses <1> to <15> characterized in that is a liquid composition at 25° C. and atmospheric pressure and comprises one or more organic solvent(s) as compound(s) according to group f), preferably one more more mono-, di-, and trivalent alcohols, and/or their mixtures as one or more compound(s) according to f).

<25> The composition according to clause <24> characterized in that one or more compound(s) according to group f) is/are ethanol, n-propanol, isopropanol, propylene glycol, ethylene glycol, benzyl alcohol, phenoxyethanol, and glycerol, and/or their mixtures.

<26> The composition according to any of the clauses <24> to <25> characterized in that the total concentration of one or more compound(s) according to group f) is in the range of 75% to 98% by weight, more preferably 80% to 95% by weight, further more preferably in the range of 85% to 92% by weight, calculated to the total weight of composition A.

<27> The composition according to any of the clauses <1> to <15> and/or <21> to <23> characterized in that composition A is a hair dyeing oil.

<28> The composition according to clause <27> characterized in that composition A has a paste-like viscosity, more preferably it has a viscosity in the range of 10,000 mPas to 500,000 mPas, more preferably in the range of 20,000 mPas to 400,000 mPas, still more preferably in the range of 30,000 mPas to 250,000 mPas, measured by cone-plate viscometry at 25° C. and atmospheric pressure.

<29> The composition according to any of the clauses <27> to <28> characterized in that the total concentration of one or more compound(s) according to group e) is 90% by weight or less, preferably in the range of 50% to 90% by weight, calculated to the total weight of composition A.

<30> The composition according to any of the clauses <1> to <29> characterized in that it comprises one or more surfactant(s) as compound according to group g), preferably selected from non-ionic surfactants, anionic surfactants, cationic surfactants, and/or amphoteric/zwitterionic surfactants, and/or their salt(s), and/or their mixtures, more preferably selected from anionic surfactants and/or their salt(s).

<31> The composition according to clause <30> characterized in that one or more compound(s) according to group g) is/are one or more anionic surfactant(s), preferably selected from ethoxylated or non-ethoxylated alkyl ether sulfate surfactants, alkyl sulfates, ethoxylated and/or non-ethoxylated alkyl carboxylates, ethoxylated or non-ethoxylated amino acid surfactants, and/or their mixtures, and/or their salts, one or more alkyl sulfate(s) preferably ethoxylated alkyl ether sulfate surfactants or mixtures thereof, and/or salts thereof, having an alkyl chain length of C₁₀ to C₂₂ and an ethoxylation degree from 1 to 50, one or more non-ionic surfactant(s) selected from alkyl glycosides, alkyl polyglycosides, ethoxylated triglycerides, ethoxylated fatty alcohols, ethoxylated fatty acid esters, and/or their mixtures, one or more cationic surfactant(s) selected from quaternary ammonium surfactants having a carbon chain length in the range of C₁₂ to C₂₂ or surfactants having a tertiary amine group and at least one alkyl chain having a carbon chain length in the range of C₁₂ to C₂₂ such as alkylamidoalkylamine surfactants, and/or their salts, and/or their mixtures, and one or more amphoteric/zwitterionic surfactants of betaine type, and/or their salt(s), and/or their mixtures.

<32> The composition according to any of the clauses <30> to <31> characterized in that the total concentration of surfactants is in the range of 0.1% to 10% by weight, calculated to the total weight composition A.

<33> The composition according to any of the clauses <1> to <32> characterized in that it comprises one or more thickening polymer(s), preferably one or more thickening polymer(s) selected from non-ionic thickening polymers and/or anionic thickening polymers, and/or their mixtures.

<34> The composition according to clause <33> characterized in that one or more thickening polymer(s) is/are a non-ionic thickening polymer, preferably a cellulose-based polymers, more preferably methylcellulose, hydroxymethylcellulose, hydroxyethylcellulose, hydroxypropylcellulose, hydroxyethyl-methylcellulose, and alkylated hydroxyl celluloses such as (C₂—C)-alkylcelluloses or cetyl hydroxyethylcellulose, an anionic thickening polymer, preferably a naturally-based anionic polymers and/or synthetic anionic polymers, more preferably xanthan gum, dehydroxanthan gum, hydroxypropylxanthan gum, carboxymethyl cellulose and starch based polymers such as vegetable starch and/or their synthetically modified derivatives such as hydroxypropyl starch phosphate, alginic acids, sodium alginates, ammonium alginates, calcium alginates, gum arabic, and guar gum, and/or an associative thickening polymer such as acrylates/steareth-30 methacrylate copolymer.

<35> The composition according to any of the clauses <33> to <34> characterized in that one or more thickening polymer is a natural anionic polymer, more preferably xanthan gum and/or dehydroxanthan gum.

<36> The composition according to any of the clauses <33> to <35> characterized in that the total concentration of thickening polymers is in the range of 0.1% to 15% by weight, preferably in the range of 0.25% to 12% by weight, more preferably in the range of 0.5% to 10% by weight, calculated to the total weight of composition A.

<37> The composition according to any of the clauses <33> to <36> characterized in that it has a viscosity in the range of 1,000 mPas to 25,000 mPas, preferably 2,000 mPas to 20,000 mPas, more preferably in the range of 2,500 mPas to 17,500 mPas, determined by cone plate viscometry at 25° C. under atmospheric conditions, preferably with a Brookfield viscometer with spindle #4, measured at 10 rpm for 1 min at 25° C. and atmospheric pressure.

<38> The composition according to any of the clauses <1> to <37> characterized in that it comprises one or more oxidizing agent(s) being solid at 25° C. and atmospheric pressure, more preferably one or more peroxide being solid at 25° C. and atmospheric pressure, still more preferably it comprises a metal peroxide such as sodium peroxide, potassium peroxide, magnesium peroxide, melamine peroxide, urea peroxide, and/or their mixtures.

<39> The composition according to clause <38> characterized in that the total concentration of one or more oxidizing agent being solid at 25° C. and atmospheric pressure is in the range of 0.1% to 50% by weight, more preferably in the range of 0.25% to 40% by weight, further more preferably in the range of 1% to 30% by weight, calculated to the total weight composition A.

The present disclosure is also directed to <40> a two-part hair dyeing composition or three-part hair dyeing and bleaching composition comprising composition A as defined in any of the clauses <1> to <39> and an aqueous composition B, optionally having a pH in the range of 1 to 6 and optionally comprising one or more oxidizing agent(s), preferably hydrogen peroxide, and optionally a bleaching composition wherein compositions A and/or B comprise(s) one or more oxidizing agent(s).

<41> The composition according to clause <40> characterized in that composition B has a pH in the range of 1 to 6, preferably in the range of 1.5 to 5, more preferably in the range of 2 to 4.5, and comprises hydrogen peroxide.

<42> The composition according to any of the clauses <40> to <41> characterized in that the total concentration of hydrogen peroxide in composition B is in the range of 0.1% to 20% by weight, more preferably in the range of 0.25% to 15% by weight, further more preferably in the range of 1% to 12% by weight, calculated to the total weight of the composition B.

<43> The composition according to any of the clauses <40> to <42> characterized in that composition B is an emulsion, thickened gel, or a combination thereof, and comprises one or more lipophilic compound(s) according to group f) and/or one or more surfactant(s) according to group g) and/or one or more thickening polymer(s) as defined in any of the clauses <33> to <35>.

<44> The composition according to any of the clauses <40> to <43> characterized in that the total concentration of compounds according to group f) in composition B is in the range of 1% to 20% by weight, preferably in the range of 2% to 15% by weight, more preferably in the range of 3% to 12% by weight, calculated to the total weight of composition B.

<45> The composition according to any of the clauses <40> to <44> characterized in that the total concentration of one or more surfactant(s) as compound according to group g) in composition B in the range of 0.1% to 10% by weight, calculated to the total weight of composition B.

<46> The composition according to any of the clauses <40> to <45> characterized in that the total concentration of thickening polymers in composition B is in the range of 0.1% to 15% by weight, preferably in the range of 0.25% to 12% by weight, more preferably in the range of 0.5% to 10% by weight, calculated to the total weight of composition B.

<47> The composition according to any of the clauses <40> to <46> characterized in that composition B has a viscosity in the range of 1,000 mPas to 25,000 mPas, preferably 2,000 mPas to 20,000 mPas, more preferably in the range of 2,500 mPas to 17,500 mPas, determined by cone plate viscometry at 25° C. under atmospheric conditions, preferably measured with a Brookfield viscometer with spindle #4.

<48> The composition according to any of the clauses <40> to <47> characterized in that the bleaching composition comprises one or more bleaching compound(s), preferably one or more persalt(s) and/or peroxy salt(s), more preferably it comprises them at a total concentration in the range of 1% to 80% by weight, calculated to the total weight of the bleaching composition.

The present disclosure is also directed to <49> a method for dyeing keratin fibers, preferably human keratin fibers, more preferably human hair, comprising the steps of:
i) providing composition A as defined in any of the clauses <1> to <39> and optionally mixing it with the composition B as defined in any of the clauses <40> to <48> to yield a ready-to-use composition having a pH in the range of 7 to 12,
ii) applying the composition A or the ready-to-use composition onto keratin fibers and leaving it for a time period in the range of 1 min to 60 min,
iii) rinsing-off the keratin fibers and optionally drying the keratin fibers.

<50> The method according to clause <49> characterized in that the ready-to-use composition is applied to keratin fibers and preferably left for a time period of 1 min to 60 min, more preferably for a time period of 5 min to 45 min, further more preferably for a time period of 10 min to 35 min, as defined in step ii).

<51> The method according to any of the clauses <49> to <50> characterized in that heat is be applied while leaving the composition A or the ready-to-use composition onto keratin fibers, preferably in a temperature range of 30° C. to 50° C.

The following examples are to illustrate the invention, but not to limit it.

### EXAMPLES

TABLE 1

| | Inv. | Inv. | Inv. | Inv. | Inv. | Comp. | Comp. | Comp. | Comp. | Comp. |
|---|---|---|---|---|---|---|---|---|---|---|
| | Ex. 1 | Ex. 2 | Ex. 3 | Ex. 4 | Ex. 5 | Ex. 1 | Ex. 2 | Ex. 3 | Ex. 4 | Ex. 5 |
| Ingredients | [% by weight] | | | | | | | | | |
| 1,4-diamino-2-methoxymethyl-benzene sulfate salt | 2.71 | 2.71 | 2.71 | 4.52 | 4.52 | — | — | — | — | — |
| 1,4-diamino-2-methoxymethyl-benzene (free base) | — | — | — | — | — | 1.66 | 1.66 | 1.66 | 2.78 | 2.78 |
| 1,3-Bis-(2,4-diamino-phenoxy)-propane 4HCl | 4.71 | — | — | — | — | 4.71 | — | — | — | — |
| 2,4-Diaminophenoxyethanol | — | 2.64 | — | — | — | — | 2.64 | — | — | — |
| 2-Amino-4-Hydroxyethyl-aminoanisole | — | — | 3.06 | — | — | — | — | 3.06 | — | — |
| 5-Amino-6-chloro-o-cresol | — | — | — | 2.84 | — | — | — | — | 2.84 | — |
| 3-Amino-2,4-dichlorophenol | — | — | — | — | 3.87 | — | — | — | — | 3.87 |

| EDTA tetrasodium salt | 2.0 |
|---|---|
| Trisodium phosphate | 55.0 |
| Corn starch | Ad 100.0 |

Color results

| | | | | | | | | | | |
|---|---|---|---|---|---|---|---|---|---|---|
| Upon preparation (ΔE) | 60 | 63 | 62 | 65 | 57 | 59 | 68 | 60 | 67 | 59 |
| Upon storage at 50° C. for 2 weeks (ΔE) | 54 | 61 | 61 | 65 | 51 | 47 | 44 | 55 | 47 | 36 |
| Upon preparation - Stored (ΔΔE) | −6 | −2 | −1 | 0 | −6 | −12 | −24 | −5 | −20 | −23 |

TABLE 2

| | Inv. Ex. | Inv. Ex. | Comp. | Comp. |
|---|---|---|---|---|
| | 6 | 7 | Ex. 6 | Ex. 7 |
| Ingredients | [% by weight] | | | |
| 1,4-diamino-2-methoxymethyl-benzene sulfate salt | 2.71 | 2.71 | — | — |
| 1,4-diamino-2-methoxymethyl-benzene (free base) | — | — | 1.65 | 1.65 |
| 2,4-Diaminophenoxyethanol | 2.64 | 2.64 | 2.64 | 2.64 |
| EDTA tetrasodium salt | 2.0 | | | |
| Sodium metasilicate | 41.0 | — | 41.0 | — |
| Tris-(hydroxymethyl)-aminomethane | — | 41.0 | — | 41.0 |

| | |
|---|---|
| Corn starch | Ad 100.0 |

Color results

| | | | | |
|---|---|---|---|---|
| Upon preparation (ΔE) | 64 | 59 | 63 | 60 |
| Upon storage at 50° C. for 2 weeks (ΔE) | 63 | 57 | 44 | 41 |
| Upon preparation - Stored (ΔΔE) | −1 | −2 | −19 | −19 |

The concentration of 1,4-diamino-2-methoxymethyl-benzene in the inventive and comparative examples in tables 1 and 2 was adjusted to be equimolar, taking the different molar masses of the free base (152 g/mol) and the sulfate salt (248 g/mol) into account.

The inventive compositions showed lower color changes upon storage of the dyeing composition in comparison to the comparative compositions, irrespective of the alkalizing agent.

### Methods

Compositions and their Storage

The compositions of table 1 were prepared by conventional mixing methods. Directly upon preparation, dyeing experiments were carried out as detailed below.

The compositions were then packaged in plastic receptacles and stored at 50° C. under 80% relative humidity for 2 weeks. The dyeing experiment was repeated.

### Hair Dyeing

Goat hairstreaks (21 cm, 2 g per bundle) were permed with a commercial perming product (2 g per streak) available under the trade name Goldwell Topform Type 1 for 20 min, rinsed, and then treated with an oxidative composition for fixing the perm for 10 min. The hair streaks were shampooed and dried.

The dyeing compositions of tables 1 were mixed with an oxidative composition comprising 3% by weight of hydrogen peroxide in a weight ratio of 1:10 to prepare ready-to-use compositions having a pH in the range of 9.5 to 10.0.

2 g of the ready-to-use compositions were applied onto the permed goat hairstreaks for 20 min at 40° C. The streaks were then rinsed off and dried. The colormetric data were obtained with a color-difference meter (Datacolor Check II Plus) in the CIE colorimetric system (L*,a*,b*). Color differences (ΔE) were calculated by the following formula.

$$Δ E = \sqrt{{( {L}_{1}^{*} - {L}_{0}^{*} )}^{2} + {( {a}_{1}^{*} - {a}_{0}^{*} )}^{2} + {( {b}_{1}^{*} - {b}_{0}^{*} )}^{2}}$$

ΔΔE is the color difference on hair between freshly colored prepared compositions and composition stored for 2 weeks at 50° C.

The following examples are within the scope of the present invention.

Inventive Example 8
Composition A

| | % by weight |
|---|---|
| 1,4-diamino-2-methoxymethyl-benzene sulfate | 0.097 |
| 5-Amino-6-chloro-o-cresol | 0.1 |
| HC Blue 18 | 0.05 |
| HC Red 18 | 0.08 |
| HC Yellow 16 | 0.02 |
| Ammonium sulfate | 10.0 |
| Diatomaceous earth | ad 100.0 |

Composition A is mixed with an oxidative composition B comprising 3% by weight of hydrogen peroxide in a weight ratio of 1:10 (A:B) to prepare ready-to-use compositions having a pH in the range of 9.5 to 10.0.

Inventive Example 9
Composition A

| | % by weight |
|---|---|
| 1,4-diamino-2-methoxymethyl-benzene sulfate | 0.097 |
| 5-Amino-6-chloro-o-cresol | 0.1 |
| HC Blue 18 | 0.05 |
| HC Red 18 | 0.08 |
| HC Yellow 16 | 0.02 |
| 2-amino-2-methylpropanol | 10.0 |
| 1,2-propylene glycol | ad 100.0 |

Composition A is mixed with an oxidative composition B comprising 3% by weight of hydrogen peroxide in a weight ratio of 1:10 (A:B) to prepare ready-to-use compositions having a pH in the range of 9.5 to 10.0.

1. An oxidative dyeing composition A for keratin fibers, comprising:
a) a salt of 1,4-diamino-2-methoxymethyl-benzene, and
c) an alkalizing agent which is at least one selected from inorganic alkalizing agents, organic alkalizing agents, salts thereof, or mixtures thereof,
wherein the salt of 1,4-diamino-2-methoxymethyl-benzene is at least one selected from a sulfate salt, a hydrogen sulfate salt, a hydrochloride salt, a hydrobromide salt, a nitrate salt, a phosphate salt, a hydrogen phosphate salt, a dihydrogen phosphate salt, a methosulfate salt, a citrate salt, a succinate salt, a tartrate salt, a lactate salt, a tosylate salt, a benzenesulfonate salt, an acetate salt, or mixtures thereof,
wherein the inorganic alkalizing agent is at least one selected from metasilicates, disilicates, carbonates, bicarbonates, phosphates, alkali or earth alkaline salts thereof, or mixtures thereof,
wherein the organic alkalizing agent is at least one selected from ammonium salts, organic alkyl and alkanol amines, or salts thereof according to the following general structure: 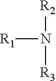
wherein R₁, R₂, and R₃ are independently selected from H, linear C₁-C₆ alkyl which may be substituted with one hydroxyl group, or branched C₃-C₁₂ alkyl or alkanol, wherein at least one of R₁, R₂, or R₃ is different from H, and
wherein a total concentration of water is 10% by weight or less, calculated to a total weight of the oxidative dyeing composition A.

2. The oxidative dyeing composition according to claim 1, wherein the salt of 1,4-diamino-2-methoxymethyl-benzene is a sulfate salt or a hydrochloride salt.

3. The oxidative dyeing composition according to claim 1, wherein a total concentration of the salt of 1,4-diamino-2-methoxymethyl-benzene is in a range of 0.01% to 65% by weight, calculated to the total weight of the oxidative dyeing composition A.

4. The oxidative dyeing composition according to claim 1, further comprising an oxidative dye precursor or oxidative dye coupler different from the salt of 1,4-diamino-2-methoxymethyl-benzene, which is at least one selected from 5-amino-2-methylphenol, 2-methyl-5-hydroxyethylaminophenol, 2,4-diaminophenoxyethanol, 2-amino-4-hydroxyethylaminoanisol, 2-methyl-5-amino-6-chlorphenol, 1,3-bis-(2,4-diaminophenoxy)-propane, 2-bis(2-hydroxyethyl)-aminotoluene, 2-amino-5-methylphenol, resorcinol, 2-methylresorcinol, 4-chlororesorcinol, 2-amino-4-chlorophenol, 5-amino-4-methoxy-2-methylphenol, 2-aminophenol, 3-amino-phenol, 1-methyl-2-hydroxy-4-aminobenzene, 3-N,N-dimethyl aminophenol, 2,6-dihydroxy-3,5-dimethoxypyridine, 5-amino-3-methylphenol, 6-amino-3-methylphenol, 2,4-diaminophenoxyethanol, 1,3-diamino-benzene, 1-amino-3-(2′-hydroxyethylamino)benzene, 1-amino-3-[bis(2′-hydroxy-ethyl) amino]benzene, α-naphthol, 4,6-dichlororesorcinol, 1,3-diamino-toluene, 4-hydroxy-1,2-methylenedioxy benzene, 1,5-dihydroxy naphthalene, 1,6-dihydroxy naphthalene, 1,7-dihydroxy naphthalene, 2,7-dihydroxy naphthalene, 1-hydroxy-2-methyl naphthalene, 4-hydroxy-1,2-methyldioxy benzene, 2,4-diamino-3-chlorophenol, 5-amino-2-methoxyphenol, 1-methoxy-2-amino-4-(2′-hydroxyethyl amino)-benzene, salts thereof or mixtures thereof.

5. The oxidative dyeing composition according to claim 1, wherein the total concentration of water in the oxidative dyeing composition A is 1% by weight or less, calculated to the total weight of the oxidative dyeing composition A.

6. The oxidative dyeing composition according to claim 1, wherein the alkalizing agent is at least one selected from sodium metasilicate, trisodium phosphate, tripotassium phosphate, or mixtures thereof.

7. The oxidative dyeing composition according to claim 1, wherein the alkalizing agent is at least one selected from monoethanolamine, diethanolamine, monoethanol methylamine, monoethanol dimethylamine, diethanolmethylamine, monoethanolethylamine, monoethanoldiethylamine, diethanolethylamine, monoethanolpropylamine, monoethanoldipropylamine, diethanolpropylamine, monoethanolbutylamine, diethanolbutylamine, trimethylamine, triethylamine, 2-amino-2-methylpropanol, tris-(hydroxymethyl)-aminomethane, salts thereof or mixtures thereof.

8. The oxidative dyeing composition according to claim 1, wherein a total concentration of the alkalizing agent is in a range 1% to 80% by weight, calculated to the total weight of the oxidative dyeing composition A.

9. The oxidative dyeing composition according to claim 1, further comprising a pulverulent excipient which is at least one selected from diatomaceous earth, kaolin, bentonite, silicates, starches, nylon powder, montmorillonit, gypsum, sawdust, perlite or mixtures thereof.

10. The oxidative dyeing composition according to claim 1, further comprising a lipophilic compound being liquid at 25° C. and atmospheric pressure.

11. The oxidative dyeing composition according to claim 1, wherein the oxidative dyeing composition is a solid composition at 25° C. and atmospheric pressure.

12. The oxidative dyeing composition according to claim 1, wherein the oxidative dyeing composition is a hair dyeing oil.

13. The oxidative dyeing composition according to claim 1, further comprising an oxidizing agent being solid at 25° C. and atmospheric pressure.

14. A two-part hair dyeing composition or three-part hair dyeing and bleaching composition comprising the oxidative dyeing composition A according to claim 1 and an aqueous composition B, optionally having a pH in a range of 1 to 6,
wherein the oxidative dyeing composition A and/or the aqueous composition B further comprises an oxidizing agent.

15. A method for dyeing keratin fibers, comprising:
i) mixing the oxidative dyeing composition A of claim 1 and with an aqueous composition B having a pH in a range of 1 to 6 to yield a ready-to-use composition having a pH in a range of 7 to 12,
ii) applying the ready-to-use composition onto the keratin fibers and leaving it for a time period in a range of 1 min to 60 min, and
iii) rinsing-off the keratin fibers and optionally drying the keratin fibers.

16. A method for stabilizing a solid dyeing composition, comprising:
adding a salt of 1,4-diamino-2-methoxymethyl-benzene to the solid dyeing composition,
wherein the solid dyeing composition comprises at least one of 4-chlororesorcinol, 2-methylresorcinol, 1,3-bis-(2,4-diamino-phenoxy)-propane, 2,4-diaminophenoxyethanol, 2-methyl-5-amino-6-chlorphenol, 2-amino-4-hydroxyethylaminoanisole, 5-amino-6-chloro-o-cresol, 2-amino-3-hydroxypyridine, 3-amino-2,4-dichlorophenol, 2,4,5,6-tetraaminopyrimidine, 2,5,6-triamino-5-pyrimidinol, salts thereof, or mixtures thereof, and
wherein the salt of 1,4-diamino-2-methoxymethyl-benzene is at least one selected from a sulfate salt, a hydrochloride salt, a hydrogen sulfate salt, a hydrobromide salt, a nitrate salt, a phosphate salt, a hydrogen phosphate salt, a dihydrogen phosphate salt, a methosulfate salt, a citrate salt, a succinate salt, a tartrate salt, a lactate salt, a tosylate salt, a benzenesulfonate salt, an acetate salt, or a mixture thereof.

17. (canceled)

18. The oxidative dyeing composition according to claim 1, wherein a total concentration of the salt of 1,4-diamino-2-methoxymethyl-benzene is in a range of 1% to 20% by weight, calculated to the total weight of the oxidative dyeing composition A.

19. The oxidative dyeing composition according to claim 1, wherein the alkalizing agent is sodium metasilicate, trisodium phosphate, tripotassium phosphate, and/or tris-(hydroxymethyl)-aminomethane.

20. The oxidative dyeing composition according to claim 10, wherein the lipophilic compound being liquid at 25° C. and atmospheric pressure is at least one selected from C12 to C22 fatty alcohols, esters of C3 to C22 alcohols with C12 to C22 fatty acids, C8 to C22 fatty acids, vegetable oils, silicones, and hydrocarbon-based products.

21. The oxidative dyeing composition according to claim 4, wherein a weight ratio of the salt of 1,4-diamino-2-methoxymethyl-benzene to the oxidative dye precursor or oxidative dye coupler is in a range of 0.2 to 5.
